(19) United States
(12) Patent Application Publication
(10) Pub. No.: US 2016/0363587 A1
(43) Pub. Date: Dec. 15, 2016

(54) Magnetic Nanoparticle Conjugate and Use Thereof

(71) Applicants: Raghuraman Kannan, Columbia, MO (US); Shreya Ghoshdastidar, Columbia, MO (US); Dhananjay Suresh, Columbia, MO (US); Danny Schust, Columbia, MO (US); Anandhi Upendran, Columbia, MO (US)

(72) Inventors: Raghuraman Kannan, Columbia, MO (US); Shreya Ghoshdastidar, Columbia, MO (US); Dhananjay Suresh, Columbia, MO (US); Danny Schust, Columbia, MO (US); Anandhi Upendran, Columbia, MO (US)

(21) Appl. No.: 15/150,262

(22) Filed: May 9, 2016

Related U.S. Application Data
(60) Provisional application No. 62/158,925, filed on May 8, 2015.
(60) Provisional application No. 62/275,100, filed on Jan. 5, 2016.

(51) Int. Cl. G01N 33/543; G01N 33/574; C12Q 1/68; G01N 33/553

(52) U.S. Cl. CPC G01N 33/54346; G01N 33/54326; G01N 33/553; G01N 33/57419; G01N 33/57423; C12Q 1/6883

(57) ABSTRACT

A composition comprising one or more antibodies covalently attached to a magnetic nanoparticle via a linker. The magnetic nanoparticle comprises a magnetic core and a non-magnetic outer surface layer. The one or more antibodies are specific for one or more target cells or one or more target biomolecules in a biological sample. The linker comprises ethylene glycol and/or thiol. The invention also provides methods for fabricating such antibody conjugated nanoparticles and procedures for their applications in selective separation/isolation of target cells or target biomolecules from biological matters.

### CROSS REFERENCE TO RELATED APPLICATIONS

This application claims priority to U.S. Provisional Application Ser. No. 62/158,925 filed May 8, 2015, and U.S. Provisional Application Ser. No. 62/275,100 filed Jan. 5, 2016, both of which are incorporated herein by reference in their entireties.

### FIELD OF THE INVENTION

The invention generally relates to the field of nanoparticles. In particular, the invention relates to antibody conjugated magnetic nanoparticles, methods of synthesizing the same, and methods of using the same in medical field.

### BACKGROUND OF THE INVENTION

Separation of biomolecules or cells of interest from a test sample is vital for analysis and quantification. Separation of biomolecules or cells may be efficiently achieved using magnetic forces to concentrate and thereby enrich the biomolecules or cells of interest from an analyte portion of a sample. Two different types of materials have been utilized to achieve magnetic separation of biomolecules and cells from the analyte. First, micron sized magnetic beads have been widely used for separating nucleic acids, cells, and biomolecules of interest. In particular, such beads typically comprises core that comprises a magnetic material and a shell surrounding the core that comprises a polymer or a glass or non-reactive metal. Second, nanoparticles comprising a magnetic element such as transition metal elements, typically oxides thereof, such as iron oxide, manganese oxide, cerium oxide.

To separate a biomolecule of interest from a test sample, a complementary sequence of DNA, peptide, or antibody is attached to magnetic beads or nanoparticles. For example, an antibody specific to a cell or antigen of interest is attached to a magnetic bead by conventional conjugation chemistry. Then the antibody/magnetic bead material is mixed with a test sample. The immobilized antibody recognizes and attaches to the molecule of interest from the sample mixture. Adequate time is allotted to facilitate binding of the two. Subsequently, the magnetic material is removed from the mixture using a magnet. Along with the magnetic material, the molecule of interest will be separated. This process enables both enrichment and separation of the molecule of interest from the mixture. The biomolecule, as well as the cell to which it may be attached, may be separated. The separated molecule and/or cell may be utilized for desired purposes, such as for quantifying the number of such molecules or cells in the sample.

Micron sized magnetic beads provide attractive opportunities for selective separation and quantification of biomolecules that are responsible for certain diseases and disease progression. U.S. Pat. Nos. 4,554,488 and 4,672,040 describe use of magnetic beads to separate DNA from samples; however, the beads disclosed therein were not nanosized and there weren't antibodies attached to the magnetic beads. U.S. Pat. No. 7,897,257 describes synthesis of magnetic beads with core metal particles to which a porous polymer matrix was attached for isolation of cells and viruses; however, the particles used were not nanoparticles, and the porous polymer matrix did not contain an antibody.

Several groups have used magnetic nanoparticles based on iron oxide for separation of biomolecules. For example, U.S. Patent Application Publication No. 2006-0286379 A1 discusses synthesis of magnetic nanoparticles with different coating compositions on the surface and biomedical applications, but their functionalization with antibodies of interest and aggregation with time were major limitation factors in their applications. U.S. Pat. No. 9,034,174 describes synthesis of a new composition of iron oxide nanoparticles for deep desulfurization; however, antibodies were not included in the surface coating. U.S. Patent Application Publication No. 2012-0070858 A1 describes a method for isolating exosomes from blood platelets using magnetic nanoparticles; however, antibodies were not employed therein.

Magnetic nanoparticles with iron oxide core and gold coating on the surface have been reported in U.S. Patent Application Publication No. 2003/0004054 A1, wherein gold was coated on the metal oxide nanoparticle for utilization as a catalyst. U.S. Patent Application Publication No. 2015/0037818 A1 describes synthesis of anisotropic iron oxide—gold nanostructures for separation and analysis of biomolecules without antibodies. U.S. Pat. No. 7,829,140 describes synthesis of iron oxide metal nanoparticles with control in core and shell thickness. However, no antibodies were used therein. Furthermore, U.S. Pat. No. 7,186,398 describes iron oxide-gold nanoparticles that possessed a large magnetic susceptibility without antibodies attached thereto.

Several efforts have been reported for controlling the size and magnetic moment of nanoparticles. However, the selective removal of molecules from biological matter is governed mainly by interaction of the antibody with the antigen of interest. In addition, retention of binding characteristics of an antibody following its attachment to a nanoparticle is believed to be quite important to ability for binding to a desired antigen. Currently, commercially available magnetic nanoparticles have several detrimental aspects that limit their usefulness—a tendency to adhere to walls of a vial or tube, poor conjugation efficiency, non-covalent attachment of antibodies to the nanoparticles, and being vulnerable to pH change. Hence, there is a need for nanoparticles that may be easily removed from the vial or tube without adhering to the walls thereof and may be easily conjugated with antibodies of choice with high efficiency. Also, there is a need for nanoparticles that allow attachment of antibodies in covalent site-specific fashion and are robust and reasonable stable to pH change. Further, there is a need to develop a synthetic method to covalently attach multiple copies of at least one selected antibody to the surface of an iron oxide-gold nanoparticle.

Prenatal screening and genetic testing are some of the most utilized and important tools in the obstetrics community. The information obtained from prenatal testing about the viability and health of an unborn child is critical to the emotional state of the parents and assists in determining clinical treatment options when needed. The ability to accurately identify genetic abnormalities as early as possible in pregnancy allows parents and physicians to make more informed decisions and reduce the expense and emotional trauma associated with detection of genetic abnormalities later in pregnancy. Down Syndrome affects even young pregnant women, and a reliable and non-invasive test is not currently available for general diagnosis. Currently, only high-risk pregnancies are normally considered for amniocentesis or chorionic villus sampling (CVS). Both of these procedures are invasive and pose a risk to both the mother and the fetus. Recently, a non-invasive cell-free fetal DNA prenatal test that requires many tubes of the mother's blood has become available (such as commercially available from Sequenom). Although this procedure is much less reliable than amniocentesis or CVS, it is appealing because it is much safer and much less expensive than the more invasive procedures such as amniocentesis or CVS. However, none of these procedures are available for use until approximately the ninth week of pregnancy. For women with low risk pregnancies, access to genetic testing is generally limited to use of one of the less reliable, non-invasive methods that are based on screening. Because of the high rate of false positives and the unreliable results obtained from the current non-invasive prenatal test, more invasive amniocentesis or CVS tests are still the predominant tests that are utilized. It is often the second trimester of pregnancy before the patient has the final answer regarding Down Syndrome indicators for the fetus. This places significant emotional stress on the patient. A patient that has suffered through the emotional trauma of a false positive and an invasive confirmatory test, or that knows of someone who has suffered such an experience, may choose to forego screening altogether. Hence, there is a need for a prenatal genetic test that would be non-invasive, would have a degree of reliability equal to or greater than the currently available invasive tests, may be performed much earlier in the pregnancy, and would be comparable in price or less expensive than current procedures.

Colorectal cancer (CRC) is one of the most common malignancies worldwide. It is readily treatable if detected in the early stages of its development. In the United States, it is the third most common cancer, and is the second leading cause of cancer death, accounting for the nation's second leading cancer-related health expenditure ($14.4 billion) and third leading cause of cancer-related lost productivity ($10.7 billion). In the US, colonoscopy for colorectal cancer screening is recommended starting at age 50 for average risk individuals, or earlier for higher risk individuals. Regular screening reduces the risk of death from colon cancer by 65%. However, only half of individuals eligible for screening undergo colonoscopy due to invasiveness of the test and associated cost. Alternatively, fecal immunochemical test (FIT) and the guaiac based fecal occult blood test (gFOBT) are the two most commonly utilized stool tests for colorectal cancer screening. Both tests detect blood in stool. gFOBT detects heme containing substances in blood, thus produces false positive because of red meat and several other dietary items. As per American Cancer Society, gFOBT requires multiple stool samples, misses most polyps and some cancer, has a higher rate of false positives, and colonoscopy becomes necessary if abnormalities are noted. On the other hand, FIT sensitivity and specificity shows significant variations among various test manufacturers. Both gFOBT and FIT are very popular among patients since both are inexpensive and noninvasive, and serve in some measure as cancer detection test. However, physicians rely on the more invasive colonoscopy exam as a cancer prevention method, due mainly to the number of false positives and inconclusive evidences associated with use of gFOBT and FIT. In 2014, the US FDA approved Cologuard® for screening fecal DNA (of hemoglobin) for detection of CRC. Clinical study confirmed that Cologuard® detected a higher percent of advanced adenomas than FIT. On the other hand, it was less accurate than FIT in identifying subjects negative for colorectal cancer. Although the test is only one year old, some studies have already demonstrated that the test misses most polyps and some cancers. In addition, it is higher in cost than the other tests. It is also uncertain how frequently the test should be performed in individuals, e.g., whether annually or biannually. Based on these reasons, it is evident that there is a definite need for accurate and sensitive sensor for early detection of colorectal cancer from feces.

Non-small cell lung cancer (NSCLC) is the leading cause for cancer related mortality rates in the US, often associated with 20-35% response rate and a ˜10 month median survival time. Currently, tissue diagnostics is performed using immunohistochemistry, FISH, and PCR for staging and treatment planning. In vivo imaging such as PET or CT is also used to detect the severity of NSCLC. NSCLC metastasize by spreading primary tumor cells to distant organs. It is possible to isolate circulating tumor cells (CTC) in patients' blood, and as the cells originate from tumor, detailed genetic evaluation about the tumor may be performed. Isolation and study on circulating tumor cells have been slowly evolving as liquid biopsy of cancer. In fact, CTC detection technique is emerging as prognostic marker to identify treatment response in NSCLC patients.

The current CTC capture methodology involves cell search technologies, predominantly relying on EpCAM expression based detection. However, with the discovery of tumor heterogeneity and the consequent impact on clinical treatment, it is important to detect patients with CTCs early on, based on their genetic alterations. Both HER-2 (2-5% mutation incidence) and EGFR (10-35% mutation incidence) overexpression have been pronounced in patient biopsies and their exclusivity in individuals have been seen as a prerequisite in chemotherapeutic selection and dose. Moreover there is no standardized process to selectively identify CTCs based on HER-2 and EGFR surface expressions. While EGFR and resistant mutations have been prominent, in the understanding of NSCLC characterization, HER-2 is relatively less explored and frequently associated with breast cancer detection. Recent studies show that HER-2 expressions correlate with metastases and disease free survival. Therefore, there is a need to develop a new sensing device or method/process to selectively identify CTCs based on markers such as HER-2 and EGFR surface expressions.

Overall, there is a need to establish the retention of affinity of antibody entities bound to the nanoparticles toward an antigen of interest and to develop an improved synthetic method of attaching multiple copies of an antibody to magnetic nanoparticles. There is also a need to separate globin from a biological sample for early detection of colorectal tumor/cancer, to separate trophoblast cells from a biological sample for early prenatal detection of chromosomal abnormalities such as Down Syndrome, and to separate circulating tumor cells from a biological sample for evaluating treatment efficacy of NSCLC.

### SUMMARY OF THE INVENTION

Provided herein is a functionalized magnetic nanoparticle comprising a nano-sized core comprising one or more magnetic atomic elements; a shell enclosing the core; one or more primary recognition elements covalently bonded to the shell via one or more linkers that comprise sulfhydryl-capped polyethylene glycol. The primary recognition elements are specific for one or more target cells or biomolecules without the aid of secondary recognitions elements bound to the target cells or biomolecules.

Also provided herein is a composition that comprises one or more antibodies covalently attached to a magnetic nanoparticle via a linker. The magnetic nanoparticle comprises a magnetic core and a non-magnetic outer surface layer. The one or more antibodies are specific for one or more target cells or one or more target biomolecules in a biological sample. The linker comprises ethylene glycol and/or thiol.

Still provided herein is a method of isolating a target cell or target biomolecule from a biological sample. The method comprises (1) contacting the biological sample with a magnetic nanoparticle conjugated with an antibody via a linker; (2) incubating the mixture to allow covalent binding of the target cell or the target biomolecule to the antibody conjugated nanoparticle; and (3) removing antibody conjugated nanoparticle that is bound to the target cell or target biomolecule using a magnet. In this method, the magnetic nanoparticle comprises a magnetic core and a non-magnetic outer surface layer. The antibody is specific for the target cell or target biomolecule, and the linker comprises ethylene glycol and/or thiol.

Further provided herein is a method of synthesizing a magnetic nanoparticle conjugate. The method comprises (1) mixing a magnetic nanoparticle with polyethylene glycol; (2) isolating PEGylated magnetic nanoparticle; (3) covalently attaching one or more antibodies to the isolated PEGylated magnetic nanoparticle, (4) isolating antibody conjugated nanoparticle, thereby obtaining a magnetic nanoparticle conjugate. In this method, the magnetic nanoparticle comprises a magnetic core and a non-magnetic outer surface layer, and the antibodies are specific for one or more target cells or one or more target biomolecules that are to be isolated from a biological sample.

Other embodiments, features, and advantages of the invention will be apparent from the following detailed description, examples, and claims.

### BRIEF DESCRIPTION OF THE DRAWINGS

The drawings described herein are for illustration purposes only and are not intended to limit the scope of the invention in any way.

FIG. 1A is a high resolution TEM image of an Au—Fe₃O₄ nanoconjugate. FIG. 1B is a high resolution TEM image of multiple such nanoconjugates of relatively uniform size.

FIG. 2A is an image of a portion of Au—Fe₃O₄ nanoconjugate. FIG. 2B illustrates the results of EDS analysis of the portion of Au—Fe₃O₄ nanoconjugate as shown in FIG. 2A, which confirms the presence of both gold and iron oxide within a single nanoparticle.

FIG. 3 is a schematic illustration of the synthesis of multiple copies of antibody conjugated Au—Fe₃O₄ nanoparticles.

FIG. 4A is a graph of absorbance as a function of wavelength for conjugated and non-conjugated Au—Fe₃O₄—PEG nanoparticles. FIG. 4B is a table showing size and zeta potential for Au—Fe₃O₄ nanoparticles, Au—Fe₃O₄—PEG nanoparticles, and Au—Fe₃O₄—PEG—Antibody nanoparticles.

FIG. 5 illustrates iron oxide nanoparticles coated with gold or inert material for subsequent conjugation.

FIG. 6 is a table showing high affinity nanomagnets candidates.

FIG. 7 illustrates various high affinity nanocubes.

FIG. 8 illustrates the process of cell separation using magnet.

FIG. 9 is a schematic illustration of separation of globin from a biological solution using Au—Fe₃O₄ nanoparticles.

FIG. 10 is a graph showing the selective capture of globin from Au—Fe₃O₄ nanoparticles conjugated with multiple copies of antibody. The control nanoparticles show minimal or negligible amount of globin capture, while Au—Fe3O4-antibody conjugate removed 13 microgram of globin from the solution.

FIG. 11 is a graph showing that an increase in concentrations of Au—Fe₃O₄—antibody conjugates increases the amount of globin separated from the solution.

FIG. 12 illustrates a linear response to globin concentration validating specificity of antibody after conjugation with HANM.

FIG. 13 is a schematic illustration of a process of separating trophoblast cells using dual iron-oxide gold nanoparticles.

FIG. 14 is a schematic illustration of a process of separating fetal trophoblast cells from a vaginal swab.

FIG. 15 contains microscopic images of normal fetal trophoblast cells and DAPI stained image.

FIG. 16 is a microscopic image confirming separation of trophoblast cells using Au—Fe₂O₃ antibody conjugates. Dark spots confirm the nanoparticles. A cluster of ˜300 trophoblast cells were separated and DAPI staining showed these dark spots are bound on the other side with trophoblast cells.

FIG. 17 is a graph that shows separation efficiency of fetal trophoblast cells compared with controls.

FIG. 18 illustrates cell counting using Cytation 3, wherein high affinity nanomagnets without antibodies attached showed no isolation of cells.

FIG. 19A illustrates isolation of JEG-3 cells at the concentration of 0.5×10⁵ cells/ml with vs. without antibody. FIG. 19B illustrates isolation of JEG-3 cells at the concentration of 0.5×10⁴ cells/ml with vs. without antibody.

FIG. 20 illustrates cell counts at different particle concentrations.

FIG. 21 illustrates cell counts at different antibody concentrations.

FIGS. 22A-22B illustrate selective isolation of JEG-3 from JEG-3+SKBR-3 mixture.

FIG. 23 shows that HANM-29 removes trophoblast cells at different temperatures.

FIG. 24 shows that HANM-29 removes JEG-3 cells.

FIG. 25 is a schematic illustration of the FeNC functionalized with either Herceptin or Cetuximab as markers.

FIGS. 26A-26C illustrate that magnetic nanoparticles (MNPs) with targeting agents capture more cells.

FIG. 27 is schematic illustration of cell sensing using magnetic nanoparticles (MNP), counting and subsequent separation of live A549 (HER2+ve; EGFR+ve) and HCC827 (HER2−ye; EGFR+ve) cells.

FIGS. 28A-28B illustrate that sensitivity increases with increasing particle concentration.

FIGS. 29A-29D illustrate an exemplary detection using nanocubes.

FIG. 30 illustrates that increasing antibody concentration on MNP surface increases selectivity of the FeNC detection.

FIGS. 31A-31B illustrate cell capture in 1× PBS.

FIGS. 32A-32B illustrate that MNP-based capture method led to enrichment of CTCs from both plasma (FIG. 32A) and serum (FIG. 32B) spiked samples.

FIG. 33A illustrates 3-fold selective capture of A549 cells compared to HCC827 cells. FIG. 33B illustrates both A549 cells and HCC827 cells adhere to HANM-CTX.

FIGS. 34A-34B illustrate 1.4×10⁶ nanoparticles/ml provided ideal separation with minimal non-specific absorption.

FIGS. 35A-35C illustrate that Zeta potential of HANM is negative and therefore would reduce non-specific absorption.

FIGS. 36A-36C illustrate quantification experiments were performed by spiking both A549 and HCC827 cells in blood plasma and serum.

FIG. 37 illustrates that HANM sensor can detect as fewer as 10 cells with high specificity.

FIG. 38 illustrates that EMT state was simulated in A549 (TGFβ1/EGF treatment) and cells were successfully captured.

### DETAILED DESCRIPTION

A composition comprising one or more recognition elements (e.g., antibodies) covalently attached to a magnetic nanoparticle via a linker is disclosed herein. Therein, the magnetic nanoparticle comprises a magnetic core and an outer surface layer or shell, the one or more recognition elements are specific for one or more target cells or one or more target biomolecules in a biological sample, and the linkers are free of fatty acids, in particular long-chain fatty acids, and comprise ethylene glycol and/or thiol.

A functionalized magnetic nanoparticle is disclosed herein, which comprises a nano-sized core comprising one or more magnetic atomic elements; a shell enclosing the core; one or more primary recognition elements covalently bonded to the shell via one or more linkers that comprise sulfhydryl-capped polyethylene glycol. The primary recognition elements are specific for one or more target cells or biomolecules without the aid of secondary recognitions elements bound to the target cells or biomolecules.

### Magnetic Nanoparticles

### Magnetic Cores

In one embodiment, the magnetic core of the nanoparticle comprises iron, iron oxide, manganese, cerium oxide, or another element or molecule that is magnetic (e.g., ferromagnetic or paramagnetic). In one embodiment of the present invention, the nanoparticles comprise a core that comprises one or more oxides of iron known to be paramagnetic (e.g., magnetite, Fe₃O₄, which is sometimes represented as FeO.Fe₂O₃, or maghemite, Fe₂O₃). In another embodiment, the core consists essentially of one or more iron oxides such that any other elements present are at what is considered to be impurity levels (e.g., less than about 1 wt %).

Layer(s) about the Cores

In one embodiment, the outer surface layer of the nanoparticle comprises one or more elements or molecules that are generally considered to be inert or safe or approved for administration to humans or animals. In certain embodiments, the outer surface layer comprises one or more elements or molecules that are generally considered to be “non-magnetic” such as gold (which is actually diamagnetic the contribution of which is negligible compared to ferromagnetic and paramagnetic) and/or PEG layers. For example, the non-magnetic outer layer comprises gold. In other embodiments, the outer surface layer comprises platinum, palladium, or a combination thereof.

### Shape(s)

The magnetic nanoparticles may be of any appropriate shape such as a tubes, rods, spheres, cubes, plates, and prisms. For example, in one embodiment the magnetic nanoparticles is sphere and having a diameter or size that is in a range of about 3 nm to about 80 nm. In another embodiment, it is a cube or alternatively, in a shape of a cube, with a dimension or size that is in a range of about 20 nm to about 30 nm.

### Size(s)

As used herein, the term “size,” with respect to nanoparticles, means nanoparticles able to pass through a sieve opening of that size. Sieve openings are square in shape and the size of the opening corresponds to the length of a side. For example, a spherical nanoparticle having a diameter less than 10 nm is able to pass through a 10 nm sieve opening. Similarly, a nanoparticle that is a rod having a length greater than 10 nm having and a diameter less than 10 nm is able to pass through a 10 nm sieve opening. Further, when referring to the size of a nanoparticle of the present invention, it is not intended to include any additional ligands, molecules, or moieties that have been placed on, attached to, or in contact with the outermost shell such as antibodies, polymers, DNA, RNA, proteins, peptides, aptamers, or any other molecular recognition elements.

In certain embodiments, the nanoparticles have a size such that they remain suspended or dispersed in a liquid or solution (without agitation), rather than settling under the influence of gravity (disregarding settling due to agglomeration). For spherical nanoparticles, in liquids having a viscosity and density about that of water, that size is typically no greater than about 100 nm. In other embodiments, including in vivo applications, the size of nanoparticles is less than about 10 nm. In certain other embodiments, including in vivo applications, the size of nanoparticles is less than about 6 nm. Unless noted otherwise, all references to size set forth herein are the average size of a multiplicity of nanoparticles.

The mathematical relationship of the nanoparticle size, magnetic field, number of ligands, and the cell can be determined in such a way that it allows greater protection but would require defining the limits that retain cell integrity (see, Kato et al., J. Mol. Cell Cardiol., 1996, 28(7): 1515-1522; incorporated herein by reference).

Fatty Acid Removal from Commercially Available Nanoparticles (NPs)

Commercially available magnetic nanoparticles (NPs) may contain fatty acids on the surface as a protecting agent. Fatty acids serve as exchange ligands with antibodies, some fatty acid molecules still partially attached to the surface even after conjugation. The importance of fatty acid removal became evident upon the following observation: after conjugation with antibody on the NPs, the fatty acid on the surface of the NPs adsorbed to the sides of the test tubes/vials and prevented it from solubilizing back to the solution. Without removing the fatty acids, the NPs would be useless. However, removal of fatty acid from the surface of NPs is challenging. Previous methodologies failed to demonstrate successful removal of the fatty acid from surface of NPs. In order to prevent to agglomeration of NPs on the sides of the tube, acetone was used to precipitate the NPs selectivity from the mixture. Free fatty acids and other surface adsorbed molecules precipitated from the 1-octadecene. The use of acetone in the ratio of 1:4 was advantageous in that excess acetone or less acetone would lead to disintegration or precipitation of the NPs. The unique combination was chosen based on the amount of fatty acid and water present in the reaction mixture. Acetone is routinely used in the art to remove water and has seldom been used to remove fatty acid.

### Linker(s)

To attach an antibody to the surface of NPs, several linkers of varying lengths were used in the art. However, the art-known designs placed the antibody far from the NPs, making it more structurally immobile and non-specific. In this study, shorter, structurally rigid PEG 200 and PEG 10000 were used for conjugating NPs with an antibody. The structural mobility of the antibody is important in increasing specificity when detecting antigen(s) of interest.

In one embodiment, the linker is ethylene glycol selected from the group consisting of monoethylene glycol, diethylene glycol, and polyethylene glycol, or a combination thereof. For example, PEG 200 and PEG 10000 can be used as the linkers.

In another embodiment, the linker is thiol selected from the group consisting of thiotic acid, monothioctic acids, dithioctic acid and trithioctic acid, or a combination thereof.

### Recognition Element(s)

Traditionally, antibody was attached magnetic microbeads via attachment of a secondary antibody to the beads. Subsequently the beads were attached to primary antibody (HLAG) at 4° C. by electrostatic attachment. In this study, one step/direct linking of HLAG antibody to the surface of the NPs was used. In doing so, the separation time was shortened from 12 hours to 2 hours. The shortened time helps to retain the structural integrity of the antibody and reduce non-specific binding, which enables quicker generation of the antibody nanoparticle conjugates.

Provided herein is a method of isolating a target cell or target biomolecule from a biological sample. Such a method comprises: (1) contacting the biological sample with one or more magnetic nanoparticle conjugated with at least one recognition element (e.g., antibodies, small peptides, small molecules, lectins, aptamers, engineered proteins, protein fragments, etc.) via a linker that is covalently bound to the surface of the magnetic nanoparticle (i.e., the outer layer or shell), wherein the linker comprises ethylene glycol, thiol, or both, and the recognition element is specific to, or has an affinity for, one or more particular target cell(s) or biomolecule(s); (2) incubating the mixture to allow covalent binding of the target cell or the target biomolecule to the recognition element conjugated nanoparticle(s); and (3) separating the recognition element conjugated nanoparticle(s), at least some of which are bound to the target cell or target biomolecule, from the non-bound portion of the sample using a magnetic field thereby isolating the target cell or biomolecule from the sample.

Specifically provided herein are gold—iron nanoparticle conjugates containing an antibody as well as methods for their preparation and procedures for their use for selective separation of globin or trophoblast cells from biological matter. The first step of the separation procedure utilizes an antigen-antibody interaction as a means to separate a molecule of interest, and thereby a cell of interest. In an embodiment, a rationally designed water-soluble iron oxide core—gold shell nanoparticle that is covalently conjugated with multiple copies of a chosen antibody and polyethylene glycol is utilized for selective separation of globin or trophoblast cells from test sample, as may be desired. An antibody nanoparticle conjugate may recognize an antigen (either globin or trophoblast) in a biological solution and may selectively bind with that molecule, and thereby selectively bind to the cell of which it is a part. In the second step of the procedure, magnetic separation of the reacted nanoparticle conjugates after sufficient time of incubation removes the antigen along with the nanoparticle conjugates. Presence of antigen among nanoparticle conjugates was confirmed by multiple analytical techniques. This method is suitable for removing minute quantities of the selected antigen. It also enables subsequent characterization and quantification of the selected antigen. For example, selective separation of fetal trophoblast cells may be achieved by choosing the proper antibody on a nanoparticle conjugate for binding to an antigen present on fetal trophoblast cells. Such separation would be useful as an aid in detecting the genetic health of a fetus. In similar fashion, nanoparticle conjugates developed in this study may be used to selectively separate minute quantities of globin from feces for early detection of colorectal cancer by proper choice of antibody chosen.

### Exemplary Targets/Uses

In one embodiment, the antibody(ies) is/are specific for a fetal trophoblast cell, a globin, or a circulating tumor cell (CTC). The applicable antibodies include, but are not limited to, EGFR antibody, Her2 antibody, EpCAM antibody, EGF-avid peptides and aptamers, and HLAG-avid peptide and aptamers.

In one embodiment, the biological sample is obtained from a placenta or a vaginal swab of a pregnant woman and the target cell is a fetal trophoblast cell. The isolated fetal trophoblast cell may be further analyzed for early detection of Down Syndrome or other chromosomal abnormalities. In one embodiment, there is provided a method of detecting Down Syndrome in a fetus by conducting a genetic test on one or more isolated fetal trophoblast cells obtained using the method of the present invention.

In one embodiment, the biological sample is a human feces sample and the target biomolecule is a human globin. The isolated human globin may be further analyzed for early detection of colorectal cancer. In one embodiment, there is provided a method of detecting colorectal cancer in human by conducting a colorimetric test on isolated human globin obtained using the method of the present invention.

In one embodiment, the biological sample is a human blood sample and the target cell is a circulating tumor cell (e.g., HER2 and EGFR positive metastatic cell). Herceptin or Cetuximab may be used as the antibody conjugated to the magnetic nanoparticle. The isolated circulating tumor cell may be further analyzed for evaluation of non-small cell lung cancer treatment. In one embodiment, there is provided a method of detecting lung cancer in a human by conducting a genetic and fluorescent immunohistochemistry test on one or more isolated circulating tumor cells obtained using the method of the present invention.

In one embodiment the conjugated magnetic nanoparticles comprise “bispecific” antibodies such that there are two more different antibodies, which are specific for different targets of a particular condition. For example, a first antibody type such as 4H84 is more effective at binding with fetal trophoblast cells within a sample obtained from a woman during the first or second months of pregnancy whereas a second antibody type such as MEMG2/G9, is more effective at binding with fetal trophoblast cells within a sample obtained from a woman during the third or fourth months of pregnancy.

Exemplary Magnetic Nanocubes Functionalize with Herceptin or Cetuximab for Detecting Circulating Tumor Cells

Also specifically provided herein is a detection device/process based on magnetic iron nanocubes (FeNC) functionalized with either Herceptin or Cetuximab as markers for circulating tumor cells (CTC). These CTC markers may be correlated with tumor heterogeneity and used to decide therapeutic targets for first line and second line treatment. This approach involves cell sensing using magnetic nanoparticles (MNP), counting and subsequent separation of live A549 (Her2+ve; EGFR+ve) and HCC827 (Her2−ye; EGFR+ve) cells from a mixture for further processing.

Herceptin conjugated MNPs captured A549 cells effectively than HCC827. This is because A549 overexpress Her2 receptors on the surface whereas HCC827 does not. On the other hand, EGFR conjugated MNPs capture both HCC827 and A549 cells equally. It is due to the fact that both these cells express EGF receptors on the surface. In this study, as low as 10 cells were isolated from the laboratory sample doped with 20 cells in swine blood. Due to the specificity of the magnetic nanocubes, the isolated cells were used to understand the genetic composition and tumor heterogeneity.

The data suggests strong correlation between number of A549 cell captured when Herceptin conjugated MNPs are used (96% difference vs HCC827), while Cetuximab conjugated MNPs pull both HCC827 as well as A549 (31% difference). It may be expected to reduce the cell capture limit to less than 10 cells in further experiments as required for patient testing. In conclusion, our results show the MNP based sensing allows both cell marker characterization as well as capture simultaneously. The nanocubes allow better characterization of HER-2 and EGFR positive metastatic cell subpopulations and provide easier prediction of tumor heterogeneity without invasive procedures.

### Synthesis of Magnetic Nanoparticle-Antibody Conjugate

Further provided is a method of synthesizing a magnetic nanoparticle conjugate. Such method comprises (1) mixing a magnetic nanoparticle with polyethylene glycol; (2) isolating PEGylated magnetic nanoparticle; (3) covalently attaching one or more antibodies to the isolated PEGylated magnetic nanoparticle; and (4) isolating antibody conjugated nanoparticle, thereby obtaining a magnetic nanoparticle conjugate. In this method, the magnetic nanoparticle comprises a magnetic core and a non-magnetic outer surface layer, and the antibodies are specific for one or more target cells or one or more target biomolecules that are to be isolated from a biological sample.

Particularly, the procedure involves mixing magnetic nanoparticles with thiol terminated PEG and careful variation of antibodies that need to be attached on the surface. In the situation that involves two different antibodies, the method analyzes the antibody that is preferentially useful in attaching with nanoparticles using covalent bond. Subsequently, that particular antibody is conjugated to nanoparticle using covalent bond. In the second step, the antibody nanoparticle conjugate is treated with a second antibody to attach it electrostatically with weak covalent bonds.

Other embodiments, features, and advantages of the invention will be apparent from the following detailed description, examples, and claims. It should be understood that the description and specific examples are intended for purposes of illustration only and are not intended to limit the scope of the invention.

### EXAMPLES

The following disclosed embodiments are merely representative of the invention, which may be embodied in various forms. Thus, specific structural and functional details disclosed herein are not to be interpreted as limiting. It should be understood that the entire disclosure of each reference cited herein is incorporated within the disclosure of this application.

### Example 1

### Synthesis of Iron Oxide Nanoparticles (FeNPs)

FeNPs are prepared by reduction of iron-oleate complex synthesis of iron oxide nanoparticles. Briefly, 4.8 gm of NaOH was dissolved in 50 ml DI water and 80 ml ethanol. To this mixture, 40 ml oleic acid was added slowly and while stirring, the pH of the solution was adjusted to 7. 140 ml hexane was then added to the solution. The resulting solution was heated to 60° C. and maintained at that temperature for 4 hours. Once the reaction was completed, the upper organic layer containing the iron oleate complex was removed and washed three times with 30 ml DI water through a separating funnel. After washing, the excess hexane was removed via rotary vacuum evaporation at 65° C. for 60 minutes. A dark red waxy solid was formed which was the iron oleate complex. Spherical iron oxide nanoparticles were synthesized using a previously published procedure but with some modifications (Zhen, et al.; J. Phys. Chem. C, 2011; 115(2): 327-334). 7.2 gm Iron Oleate Complex was added to 1.28 ml oleic acid and 50.69 ml 1 octadecene. The solution was heated to 300° C. and kept at that temperature for 30 minutes. The solution was then degassed under nitrogen atmosphere for 15 minutes. The resulting dark brown solution was collected and excess of acetone was added to precipitate the Fe₃O₄ solution from 1 octadecene. The Fe₃O₄ solution was centrifuged at 5000 g for 20 minutes. The pellet was collected and dissolved in chloroform. The step was repeated two more times and the final product was dissolved in chloroform.

### Example 2

### Synthesis of Iron Oxide Gold Nanoparticles (AuFeNPs)

800 μl (0.9 gm) iron oxide solution was added to 10 ml 1 Octadecene followed by addition of 1.3 gm 1, 2 hexadecanediol. The solution was heated to 100° C. and kept at that temperature for 1 hour. Gold oleylamine solution was prepared by adding 40 mg HAuCl₄ to 5 ml octadecene and 1 ml oleylamine and sonicating for 30 minutes. The iron oxide solution was further heated to 140° C. and gold oleylamine solution was added drop wise. The temperature of the solution was maintained at 140° C. for 15 minutes before increasing the temperature to 200° C. The solution was kept at 200° C. for 20 minutes and then cooled to room temperature. The Au@Fe nanoparticles were synthesized in a polyol solution which renders the nanoparticles not very easily accessible for functionalization. It is necessary to bring the nanoparticles in an aqueous phase for ease of functionalization with Peg or antibody. 1 ml of AuFe was taken in a glass vial to which 4 ml of acetone was added to precipitate the nanoparticles out of 1 octadecene. The process of precipitation was repeated 2 times with 2 ml acetone. The upper layer was removed and 1 ml 25% w/v aqueous TMAOH (surfactant) was added to the precipitated nanoparticles. The solution was sonicated for some time and then magnetically separated. The pellet was dissolved in 1 ml TMAOH by sonication. The solution was kept in front of a magnet. The above steps were repeated two times. The pellet was then dissolved in 2 ml of 7 mg/ml trisodium citrate solution and sonicated for 10 minutes. The solution was kept in front of a magnet for magnetic separation. The pellet was then dissolved in water and magnetically separated. This step was repeated two times. The final Au@Fe pellet was dissolved in 1 ml DI water and stored at room temperature.

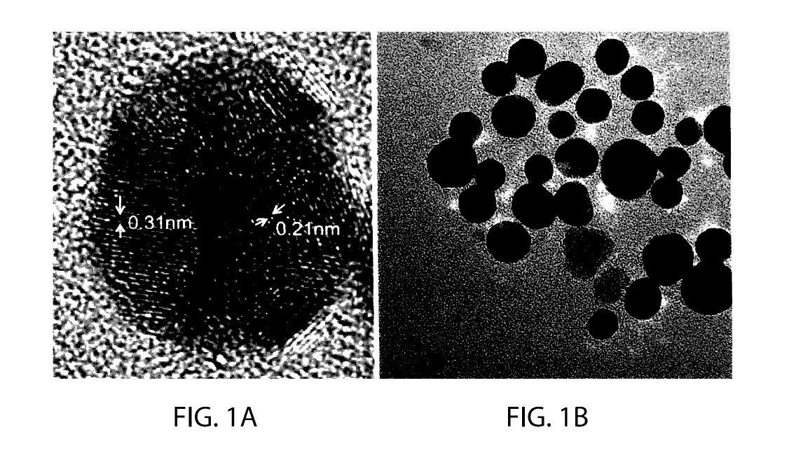

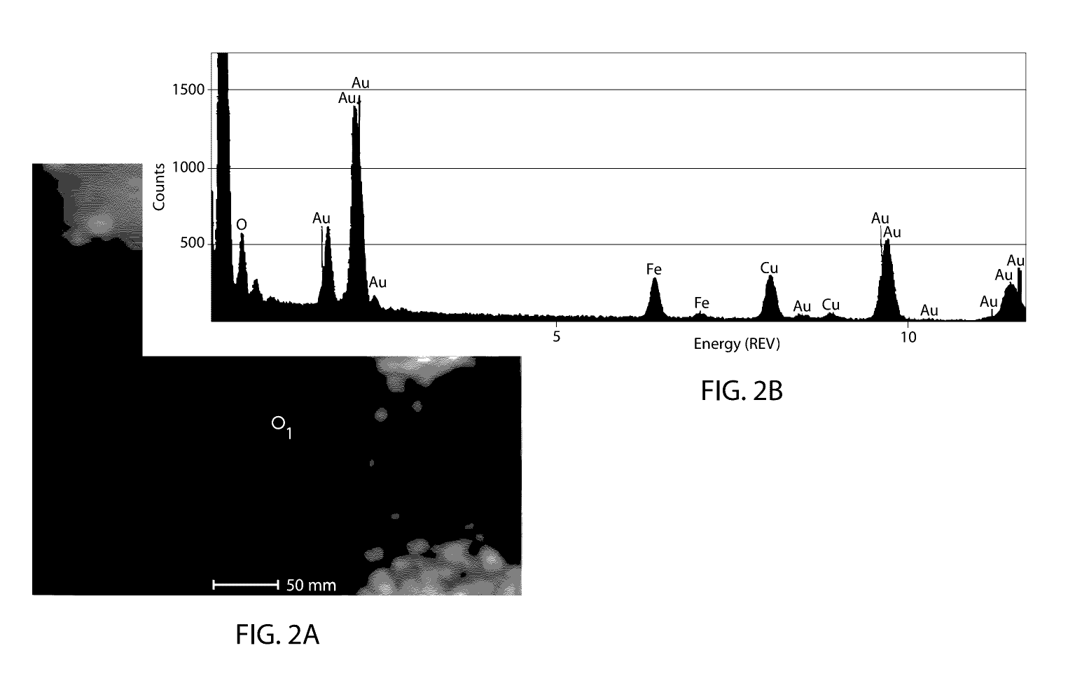

High resolution TEM of Au—Fe₃O₄ nanoconjugates confirmed the alloy of two nanoparticles together and the TEM image confirmed the uniform size of the nanoparticles (see, FIGS. 1A-1B). EDS analysis of Au—Fe₃O₄ nanoconjugates confirmed the presence of both gold and iron oxide within a single nanoparticle (see, FIGS. 2A-2B).

### Example 3

### Synthesis of PEGylated Iron Oxide Gold NP (PEG(COOH)Au@FeNP)

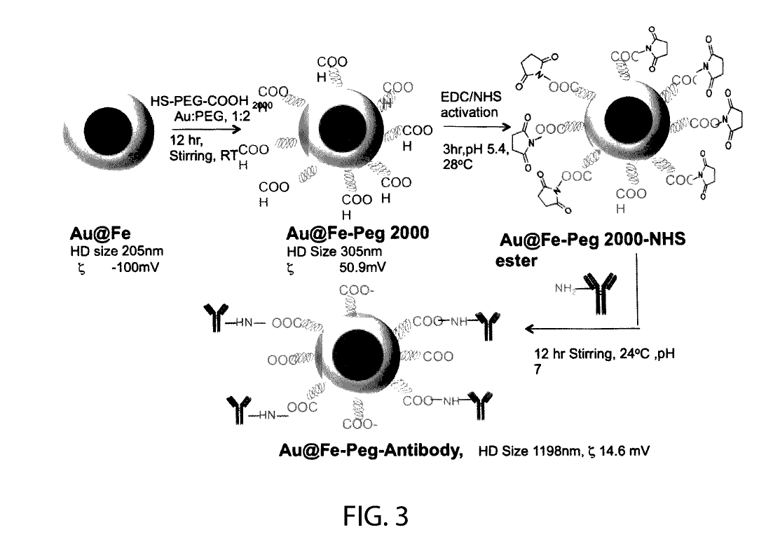

Conjugation of Au@Fe to the antibody requires the functionalizing of Au@Fe nanoparticles with polyethylene glycol (PEG; see, FIG. 3). The nanoparticles were functionalized with CM Thiol Peg 2000. 500 μl of Au@Fe solution was taken in a glass vial, to which 3 mg Thiol Peg 2000 dissolved in 500 μl DI water was added. The solution was stirred in a thermomixer at 28° C. for 5 hours to overnight. The PEGylated solution was purified by magnetic separation. 500 μl PEGylated Au@Fe nanoparticles were magnetically separated.

### Example 4

### Synthesis of Iron Oxide Gold Antibody Conjugate: (COV(Ab)Au@FeNP)

To the pellet 8 mg EDC and 8 mg NHS in 40 μl MES buffer each was added and the pellet was dissolved. The pH of the solution was adjusted to 5.8. The Au@Fe-Peg was activated for 3 hours at 28° C. 4 μl of 10.6 mg/ml anti-Human Hemoglobin IgG (polyclonal Ab) was added to 200 μl PBS solution which was added to the activated Au@Fe-thiol Peg 2000 and was stirred overnight in a thermomixer at 24° C. (see, FIG. 3). The antibody conjugated nanoparticle was purified by magnetic separation.

### Example 5

### Electrostatic Conjugation of Antibody to AuFe Nanoparticle

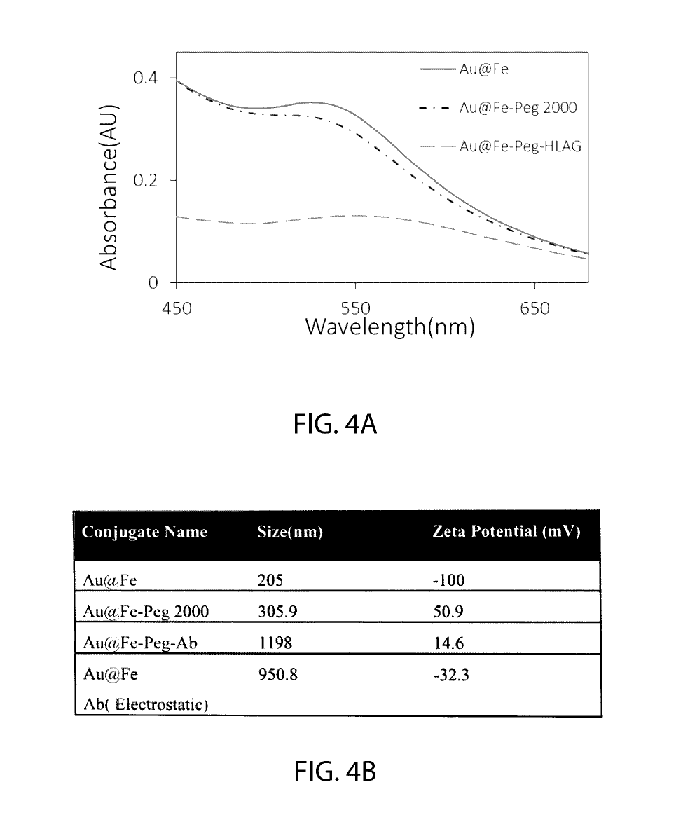

1 ml Au@Fe solution was taken in a glass vial. 500 μl 1× PBS solution was added. The pH was recorded to be 7. 20 μl (10 μg) of Mouse Anti-Human HLAG antibody was added and the solution was shaken in a thermo-mixer at 25° C. overnight. Unconjugated antibody was removed by magnetic separation and the amount of protein conjugated was estimated by Bradford assay (see, FIGS. 4A-4B).

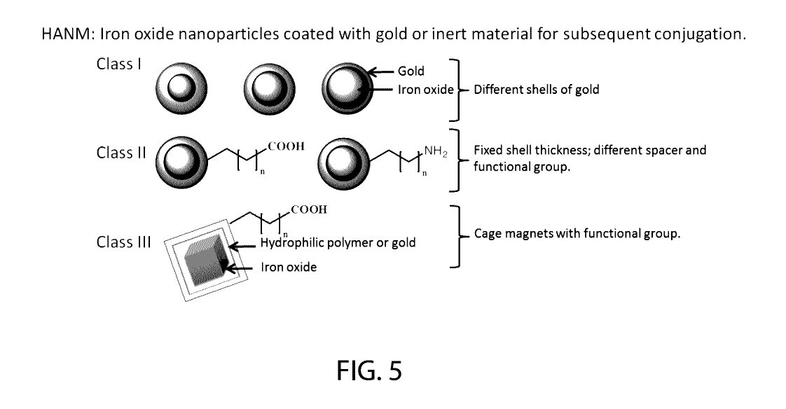

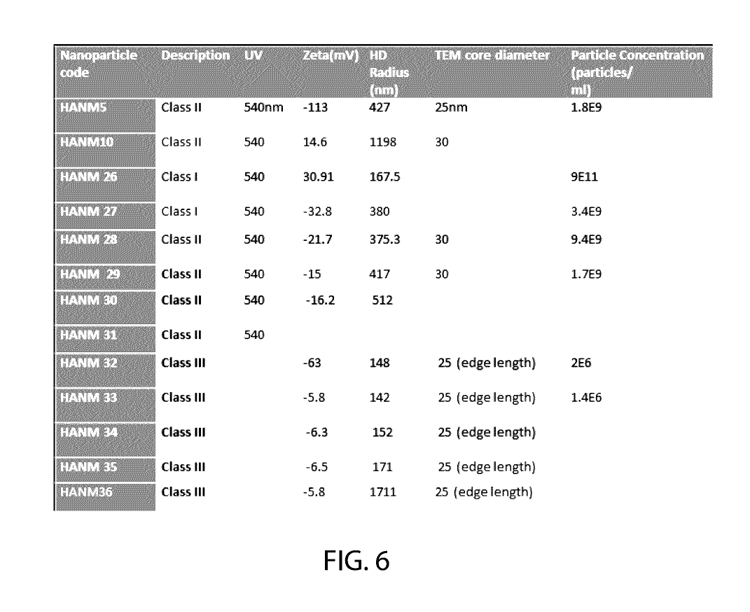

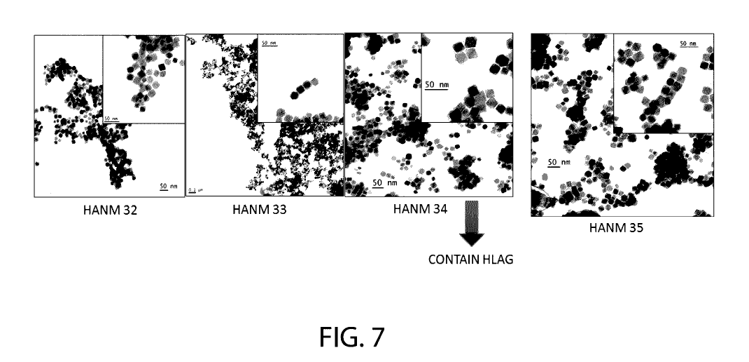

FIGS. 5-7 shows structures of high affinity nanomagnets (HANM), various HANM candidates, and characterization thereof.

### Example 6

### Cell Separation using Magnet

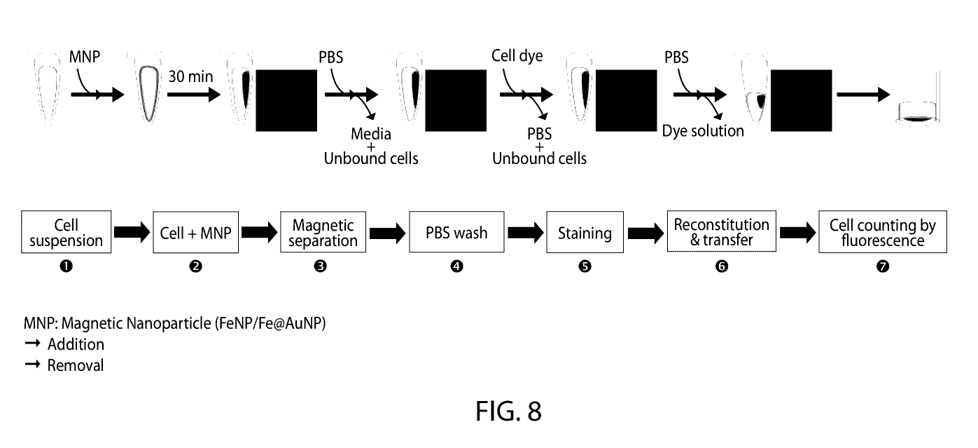

Target cells can be separated/isolated from a biological matter using the magnetic nanoparticles as shown in FIG. 8.

### Example 7

### Hemoglobin Detection Assay with AuFe-Ab Conjugate

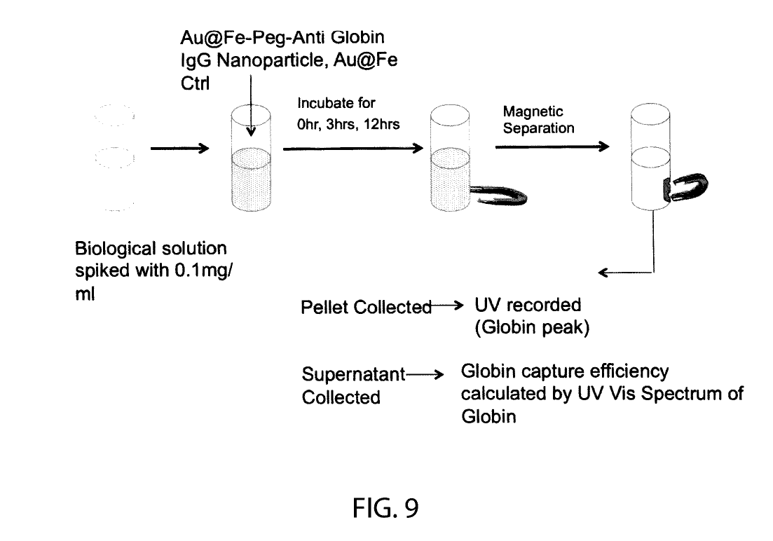

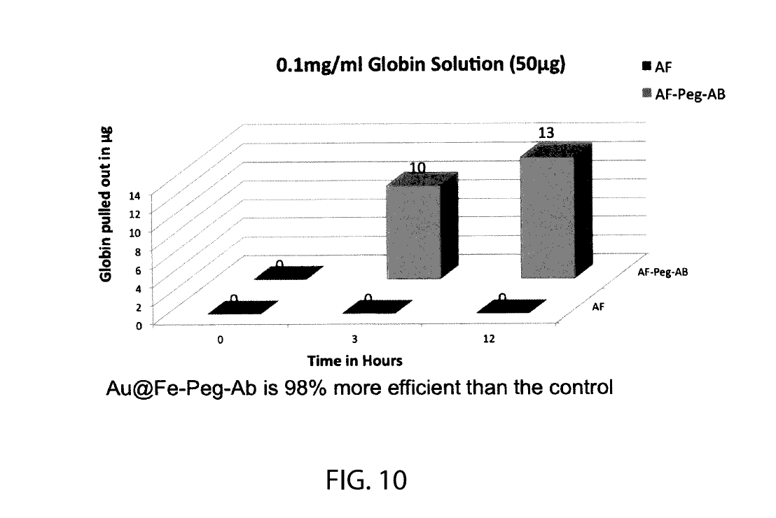

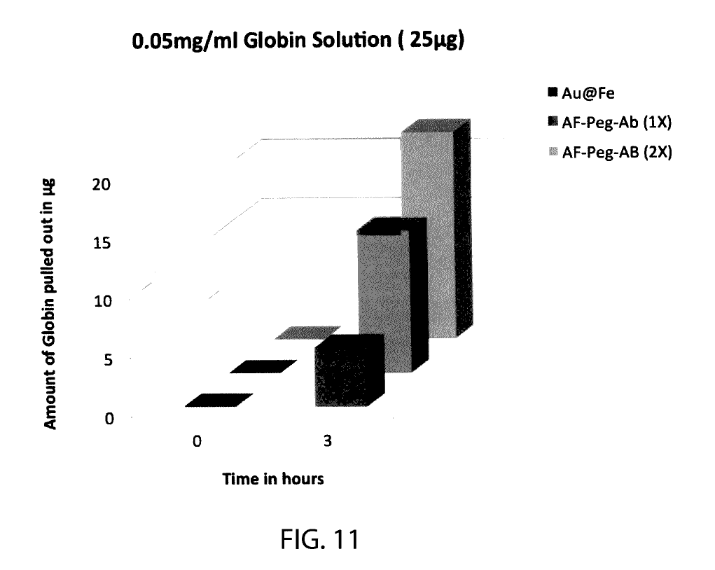

To establish the target specificity of the antibody conjugated nanoparticles, the antibody conjugated nanoparticles were incubated with 500 μl of 0.25 mg/ml and 0.1 mg/ml hemoglobin solution for different time points. The Au@Fe-anti Globin Ab nanoparticles attached to the hemoglobin molecules, and the globin-Au@Fe-Ab moiety was then separated from the globin solution using a magnet. Au@Fe nanoparticles were used as a control. Some nanoparticles were incubated for 0.5 hours at room temperature, while others were incubated for 3 hours at room temperature. The magnetically separated nanoparticles were then analyzed by spectrophotometry. Hemoglobin has an absorbance peak at 410 nm. Bradford assay was performed to quantify the amount of globin present in the supernatant after magnetic separation. The measured value of globin was subtracted from the initial globin concentration to analyze the amount of globin pulled out by the Au@Fe-Ab conjugate (see, FIGS. 9-11). It showed that the maximum amount of globin was being pulled out after incubating the nanoparticles in the globin solution for 3 hours.

500 μl of 0.1 mg/ml Hemoglobin solution in DI Water was taken in a glass vial. 200 μl of AuFe-Antibody was added. In another glass vial 200 μl of Au@Fe was added. The solutions were kept in room temperature for 3 hours. After 3 hours, the vials were kept in front of a block magnet. The pellets were collected and washed with 200 μl DI water and magnetically separated. The supernatant was collected for Bradford assay and the pellet was dissolved in 200 μl DI water and UV-Vis spectra were recorded.

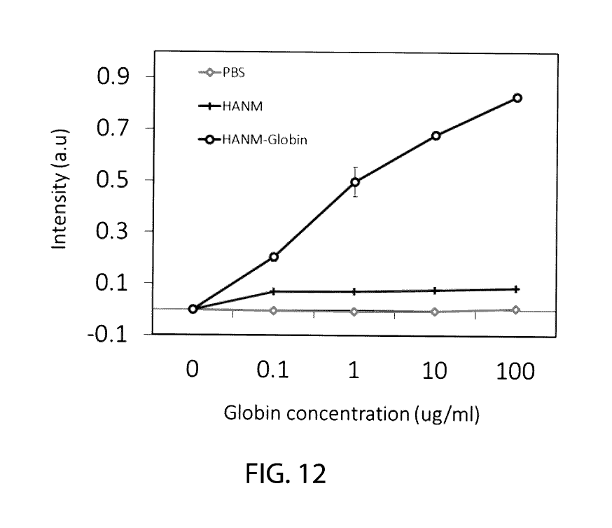

Also, when high affinity nanomagnets (HANM) conjugated with antibody specific to globin, such antibody acts as a surrogate in the present study. ELISA results showed a linear response to globin concentration validating specificity of antibody after conjugation with HANM (see, FIG. 12)

### Example 8

### Trophoblast Cell Detection Assay with AuFe-Ab Conjugate

### JEG-3 Cell Culture

JEG-3 cells in T 25 flask were trypsinized and dislodged from the flask in a single cell suspension with RPMI media. The suspended cells were centrifuged at 2000 rpm for 6 minutes. The supernatant was removed and the pellet was re-suspended in RPMI media. The cells were counted with Countess Cell counter. The cells were stained with C10446 dye (Red) and diluted to make a cell concentration of 10⁵ cells/ml.

### VS Cell Extraction

The swabs from non-pregnant women were stored in 1× PBS and were centrifuged at 2000 rpm, for 15 minutes, 3 times till all the cells were collected in a pellet. The cells were suspended in 1× PBS and centrifuged at 2000 rpm for 8 minutes. The cells were suspended in 1 ml 1× PBS and were counted. The cells were stained with Hoechst 3342 dye (Blue) and diluted to make a concentration of 10⁵ cells/ml.

### Specificity of MNP-13 (JEG-3+VS Cells)

100 μl of Jeg 3 cells (10⁴ cells) was added to 100 μl of 10⁴ VS cells. 200 μl of MN-13 (Au@Fe-MEM G9) and MNP-12 (Au@Fe-HLAG) were added separately to the cell mixture. The cells were incubated at 37° C. for 2 hours. The cells were vortexed every 30 minutes. After 2 hours, the cells were kept in front of a Neomydium block magnet for 15 minutes. The supernatant was removed and the pellet was suspended in 200 μl 1× PBS and kept in front of magnet for 15 minutes. This step was repeated 3 more times. The final pellet was suspended in 1× PBS and transferred to 96-well plate for imaging with Cytation 3.

### Trophoblast Cell Detection

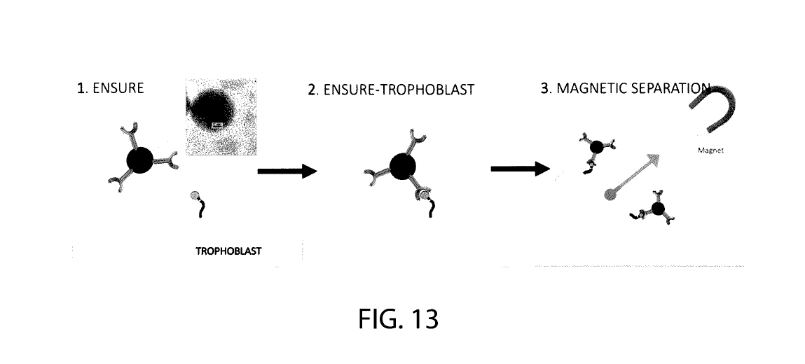

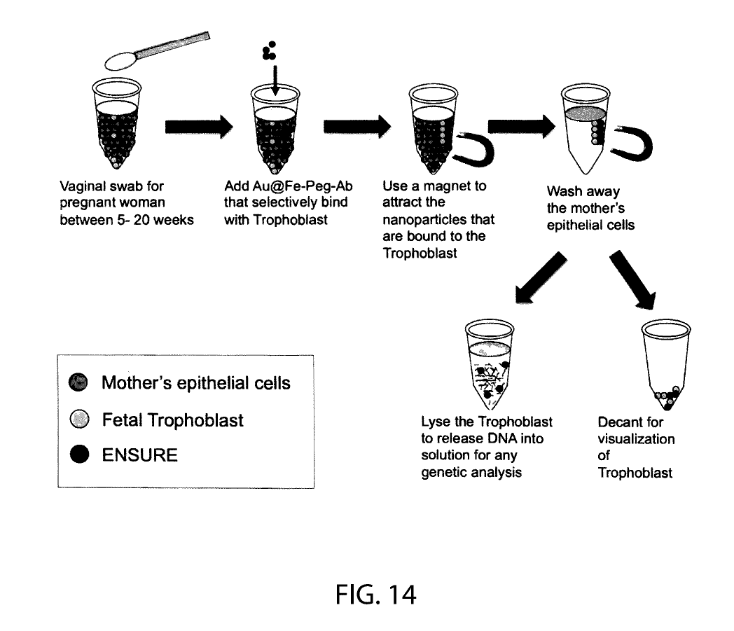

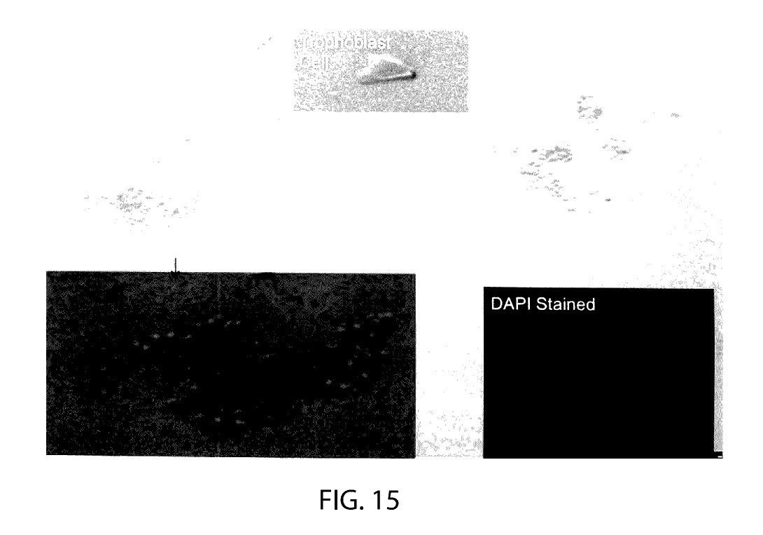

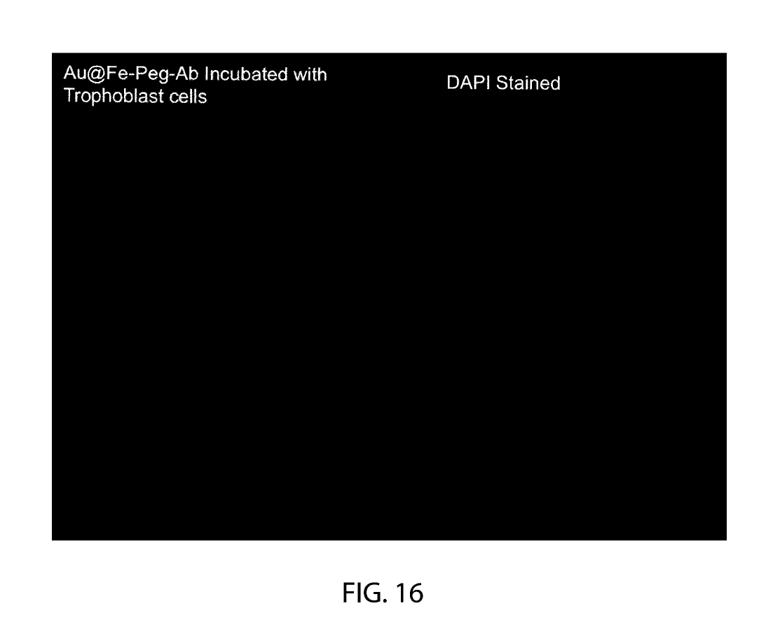

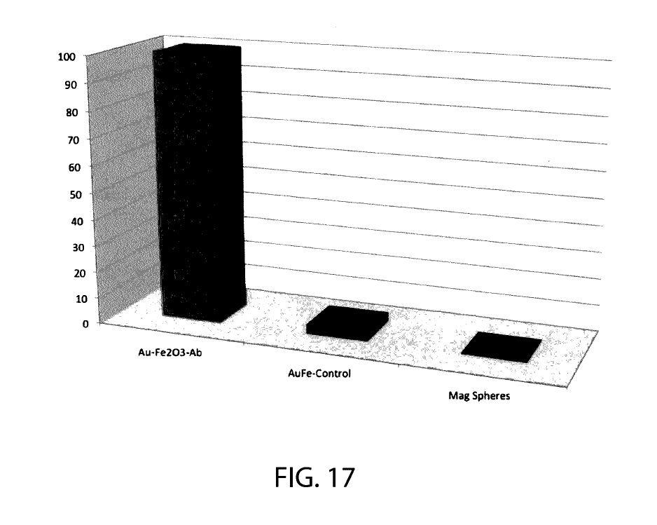

Trophoblast cells were separated from a biological sample for, e.g., a vaginal swab, using dual iron-oxide gold nanoparticles (see, FIGS. 13-14). Microscopic images of normal fetal trophoblast cells and DAPI stained image are shown in FIGS. 15-16. Compared to controls, fetal trophoblast cells were separated at a much higher efficiency (see, FIG. 17).

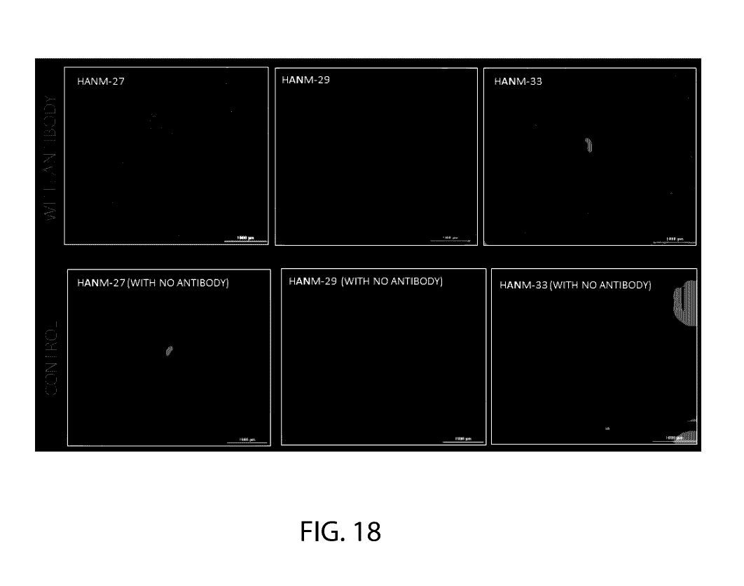

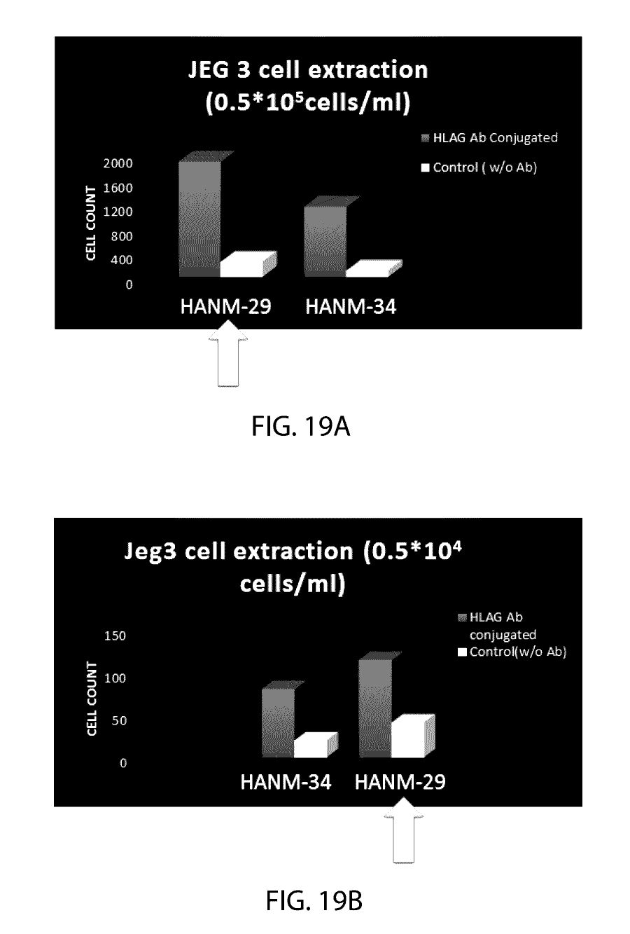

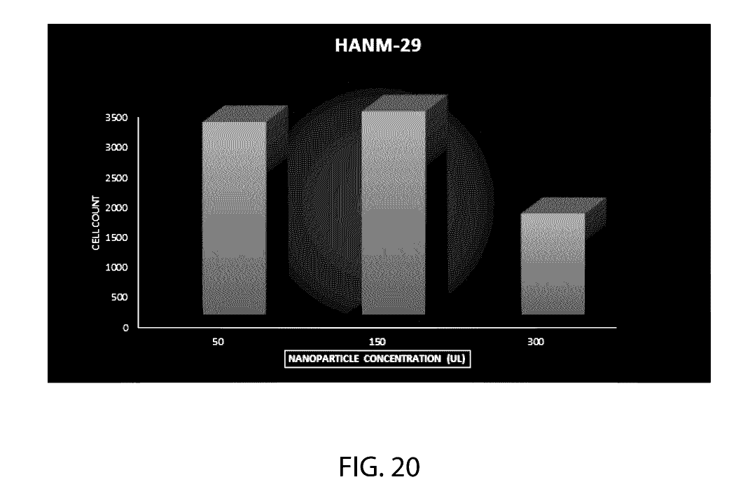

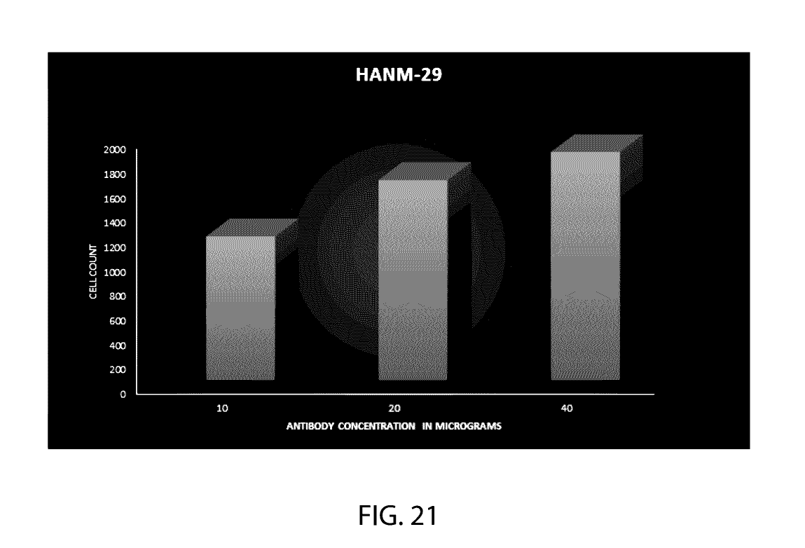

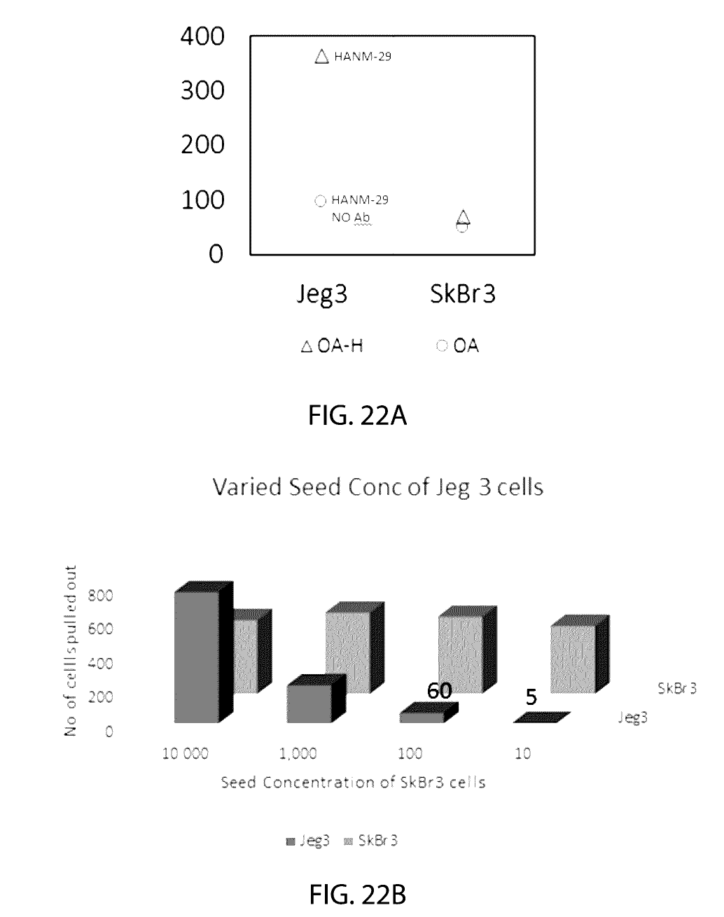

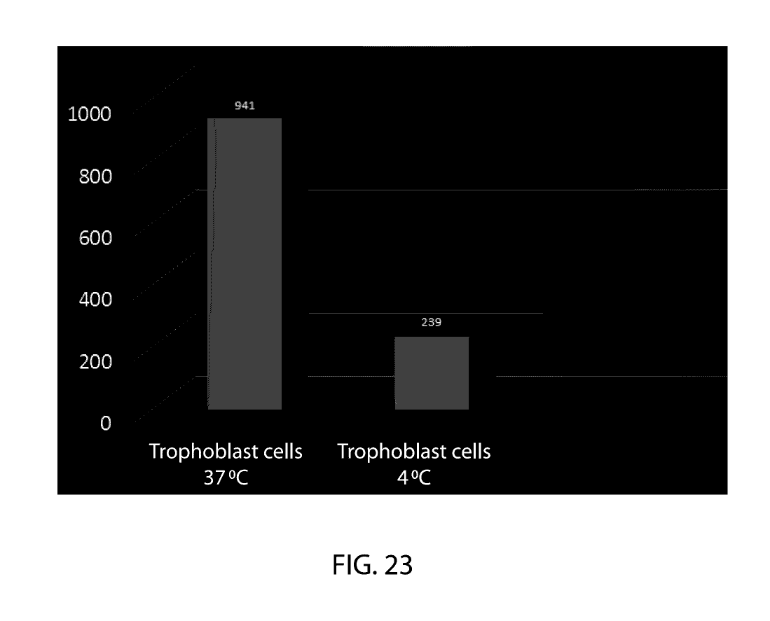

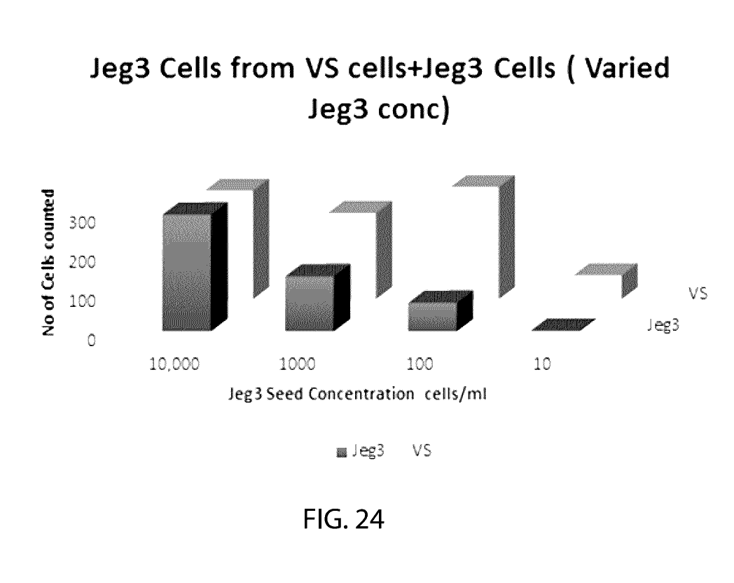

Results of isolation of JEG-3 cells from culture mixtures are shown in FIGS. 18-24.

### Example 9

### FeNC Functionalized with Preselected Markers and Uses in Detecting CTC

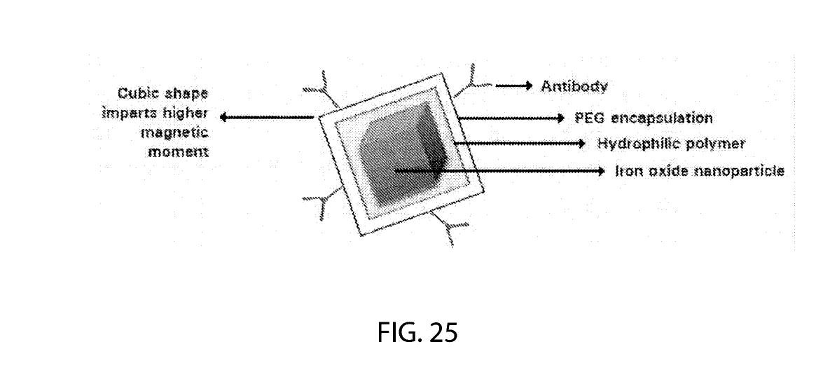

Magnetic iron nanocubes (FeNC) were functionalized with either Herceptin or Cetuximab as markers for circulating tumor cells (CTC). These CTC markers are correlated with tumor heterogeneity and may be used to decide therapeutic targets for first line and second line treatment (see, FIG. 25, for the schematic illustration of functionalizing FeNC with either Herceptin or Cetuximab as markers).

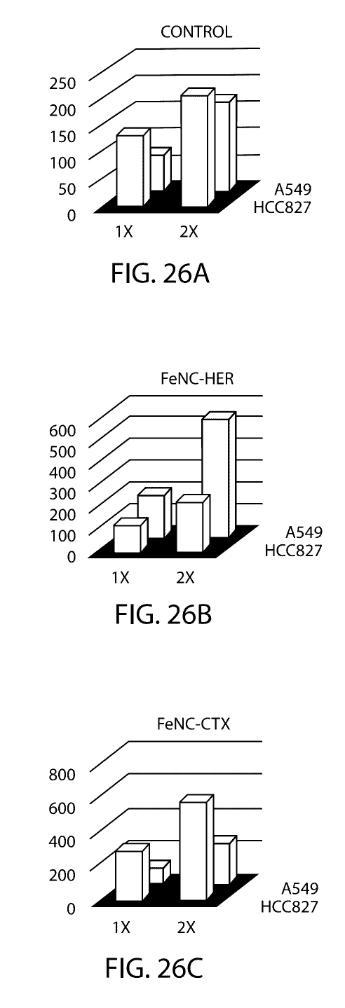

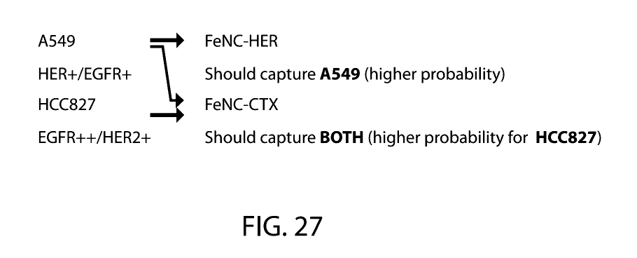

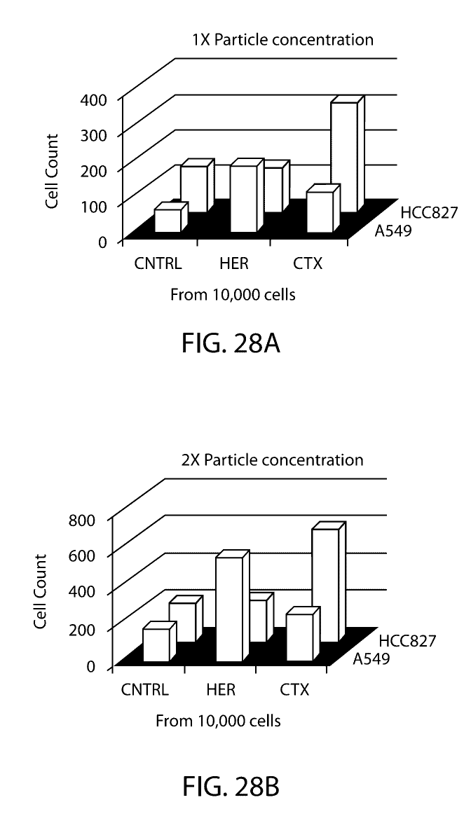

This approach involves cell sensing using magnetic nanoparticles (MNP), counting and subsequent separation of live A549 (HER2+ve; EGFR+ve) and HCC827 (HER2−ye; EGFR+ve) cells from a mixture for further processing (see, FIG. 27). HER2 (2-5% mutation incidence) and EGFR (10-35% mutation incidence) overexpression have been detected in patient biopsies and seen as a prerequisite in chemotherapeutic selection and dose. It is shown that MNPs with targeting agents capture more cells (see, FIGS. 26A-26C). It is further shown that the sensitivity increases with increasing particle concentration (see, FIGS. 28A-28B).

In particular, magnetic iron oxide nanoparticles (˜50 nm in diameter) that target HER-2 and EGFR receptors were synthetized, which were magnetic, stable in serum solutions for extended periods of time. The magnetic nanoparticles (MNPs) were subsequently incubated with cells in 1× PBS for 3 hours for receptor binding at 37° C. after which particles bound to cells were magnetically separated with a pull force of 57 lbs. Cells were then washed and counted using an automated algorithm. The whole process was optimized to a minimum cell population of 100 and may be theoretically reduced to a 2-hour process which makes this extremely effective for clinical evaluations and straightforward. Similar protocol was used when the cells were spiked in blood plasma and captured.

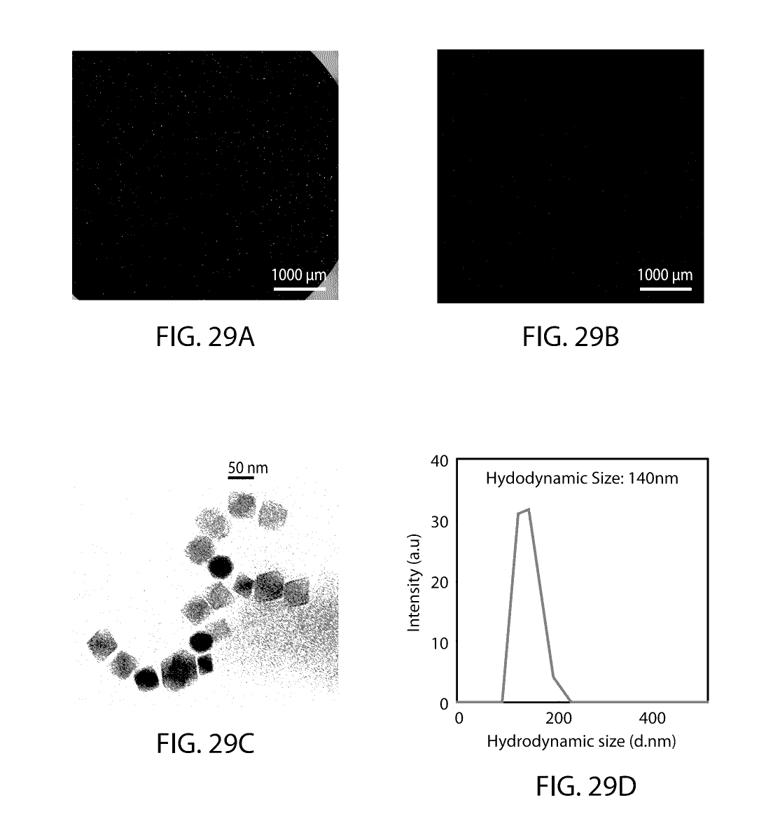

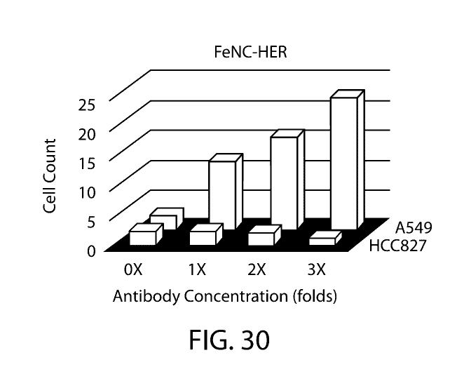

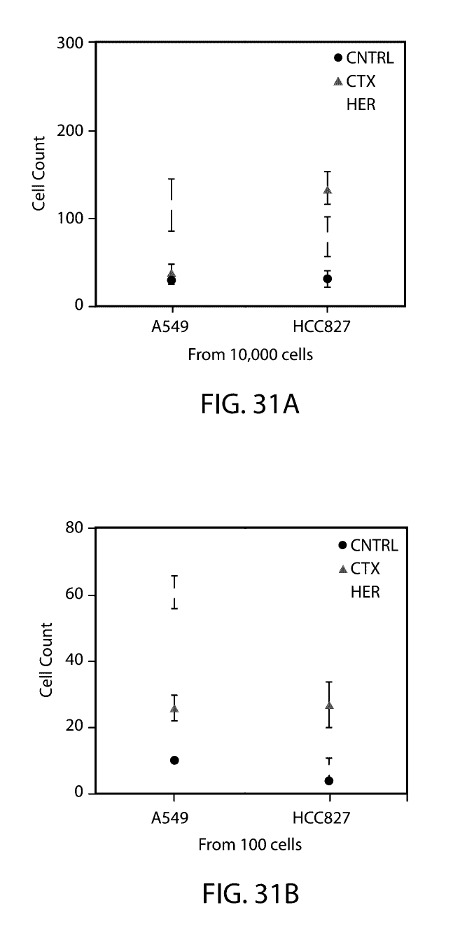

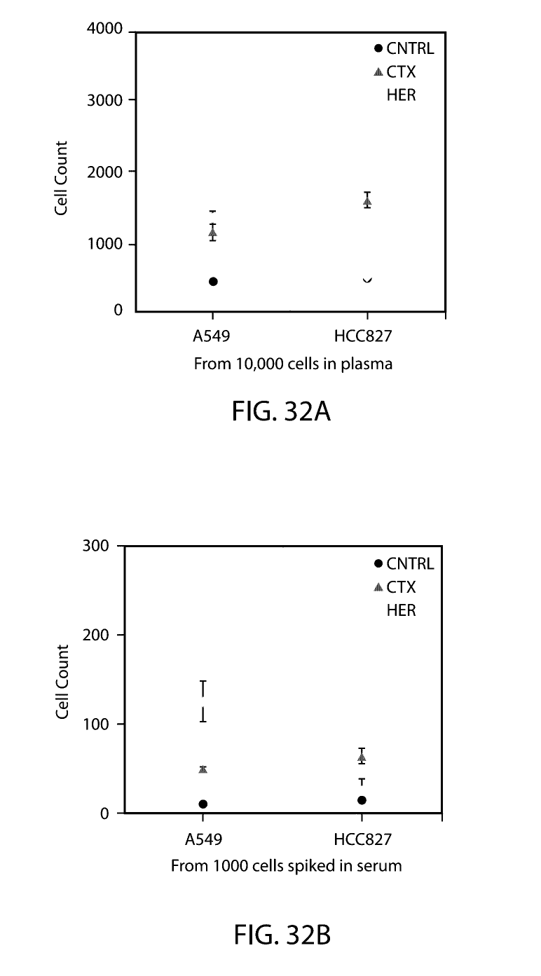

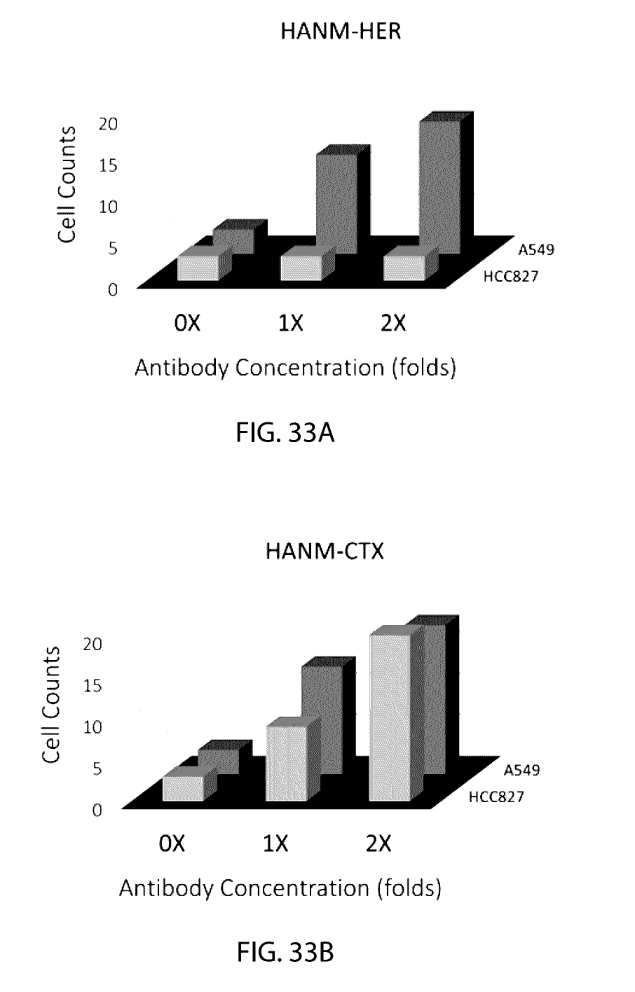

An exemplary detection using nanocubes is shown in FIGS. 29A-29D. In particular, FIGS. 29A-29C demonstrate the uniform size of iron nanocubes that are conjugated with antibody for selective removal of cell of interest. TEM measurements show uniform size nanocubes were obtained and the particles exhibit an edge length of 20 nm. Sonication and dilution yielded individual nanoparticles for further applications. FIG. 29D illustrates the hydrodynamic size of nanocubes obtained after conjugation with antibody. Zeta potential of high affinity nanomagnets (HANM) is negative and therefore would reduce non-specific absorption. It is also shown that increasing antibody concentration on MNP surface increased selectivity of the FeNC detection (see, FIG. 30). FIGS. 31A-31B illustrate cell capture in 1× PBS. Inset graph represent construct specificity towards particular cell lines, and FIG. 32 illustrates MNP-based capture method led to enrichment of CTCs from both plasma/serum spiked samples. In particular, A549 cells expressed Her2 on the surface whereas HCC827 cells did not. FeNC-HER was anticipated to selectively remove A549 cells and not HCC827 cells. As anticipated, a 3-fold selective capture of A549 cells compared to HCC827 cells was observed (see, FIGS. 33A-33B). On the other hand, FeNC-CTX was treated with a mixture of A549 cells and HCC827 cells. This nano magnet should have high affinity for EGFR. Both A549 and HCC827 cells have high degree of EGFR on the surface. Therefore, it was anticipated that both the cells would adhere to HANM-CTX. The results indicated a similar trend between both cells (see, FIGS. 33A-33B).

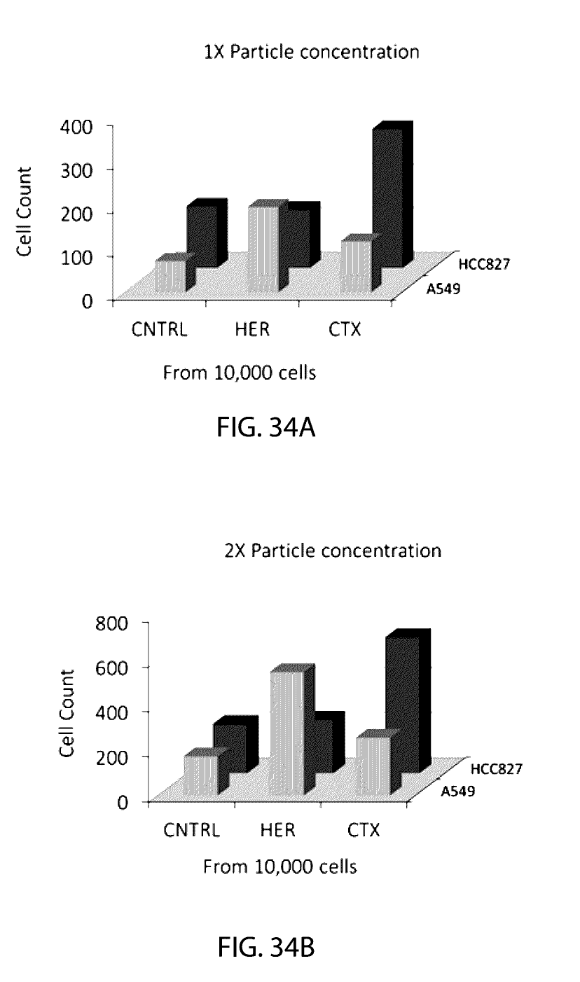

The conditions including particle concentration and incubation time were optimized for cell separation using HANM. It is shown that the concentration of 1.4×10⁶ nanoparticles/ml provided ideal separation with minimal non-specific absorption (see, FIGS. 34A-34B).

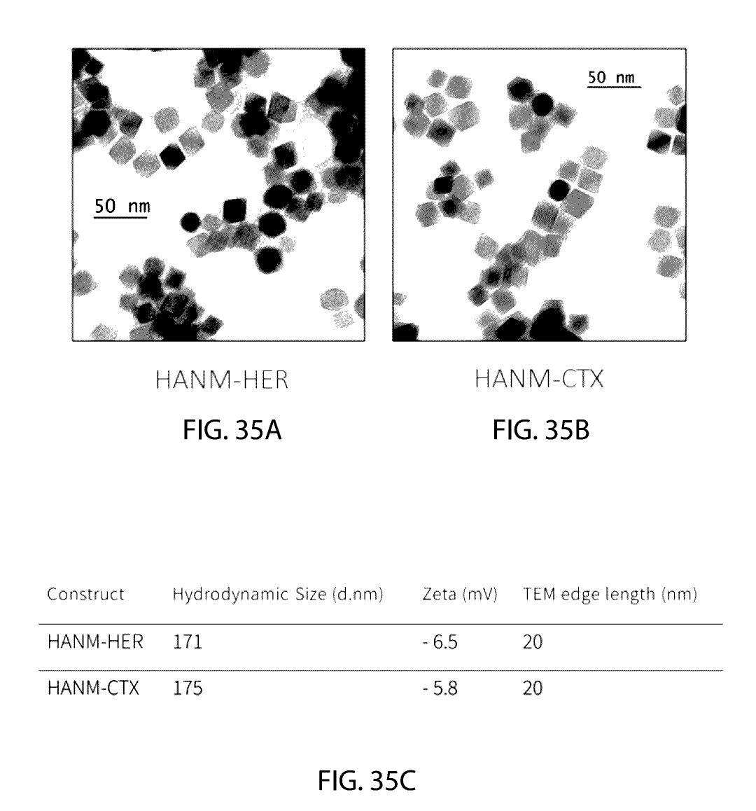

HANM was characterized using TEM measurements (see, FIGS. 35A-35C); uniform size nanocubes were obtained and the particles exhibit an edge length of 20 nm. Sonication and dilution yielded individual nanoparticles for further applications. Zeta potential of HANM is negative and therefore would reduce non-specific absorption.

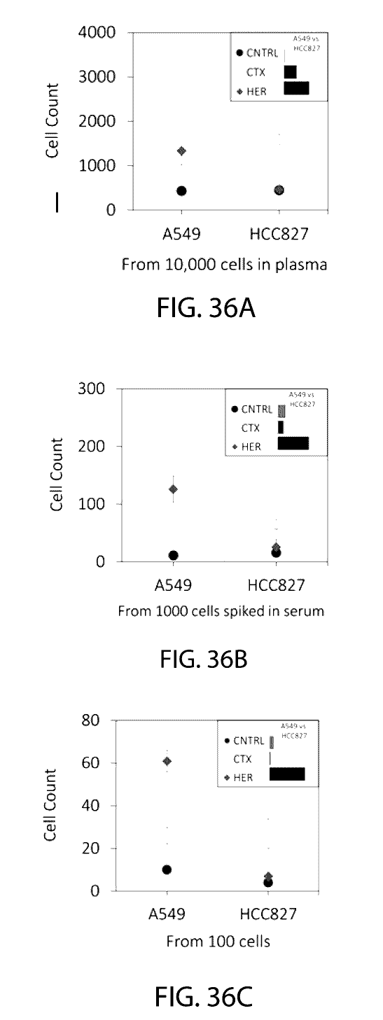

Quantification experiments were performed by spiking both A549 and HCC827 cells in blood plasma and serum. HANM-HER showed high specificity in capturing and isolating A549 cells (HER2+ve) with a selective difference of 96% (see, FIGS. 36A-36C, inset graphs represent differential specificity of each construct). HANM-CTX captured cells based on their EGFR expression with a relative difference of 31%. That is, using HER2 markers, EpCAM and cytokeratin negative cells can be isolated.

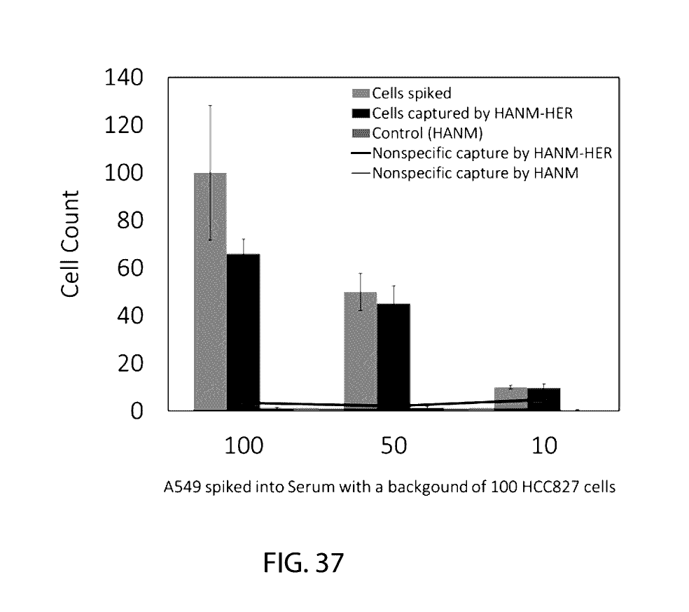

HANM sensor can detect as fewer as 10 cells with high specificity as shown in FIG. 37.

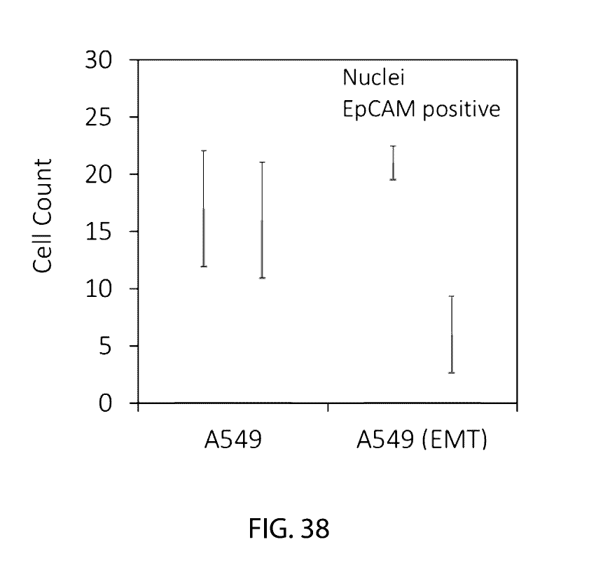

Further, EMT state was simulated in A549 (TGFβ1/EGF treatment) and cells were successfully captured. EMT status was validated on the basis of cytokeratin markers 4, 5, 6, 8, 10, 13 and 18. Cells were quantified using immunostaining (see, FIG. 38).

Having illustrated and described the principles of the present invention, it should be apparent to persons skilled in the art that the invention may be modified in arrangement and detail without departing from such principles. Those skilled in the art will recognize, or be able to ascertain using no more than routine experimentation, many equivalents to the specific embodiments of the invention described herein. Such equivalents are intended to be encompassed by the following claims.

All publications and published patent documents cited in this specification are incorporated herein by reference to the same extent as if each individual publication or patent application was specifically and individually indicated to be incorporated by reference.

What is claimed is:

1. A functionalized magnetic nanoparticle comprising:
a nanosized core comprising one or more magnetic atomic elements;
a shell enclosing the core;
one or more primary recognition elements covalently bonded to the shell via one or more linkers that comprise sulfhydryl-capped polyethylene glycol, wherein the primary recognition elements are specific for one or more target cells or biomolecules without the aid of secondary recognitions elements bound to the target cells or biomolecules.

2. A composition comprising one or more antibodies covalently attached to a magnetic nanoparticle via a linker, wherein said magnetic nanoparticle comprises a magnetic core and a non-magnetic outer surface layer, wherein said one or more antibodies are specific for one or more target cells or one or more target biomolecules in a biological sample, and wherein said linker comprises ethylene glycol and/or thiol.

3. The composition of claim 2, wherein said magnetic core comprises iron, iron oxide, manganese, cerium oxide, or another element that possesses magnetic property.

4. The composition of claim 3, wherein said magnetic core comprises iron or iron oxide.

5. The composition of claim 2, wherein said non-magnetic outer surface layer comprises gold, platinum, or palladium.

6. The composition of claim 5, wherein said non-magnetic outer layer comprises gold.

7. The composition of claim 2, wherein said magnetic nanoparticle is in a shape of sphere and has a diameter of between about 3 nm and about 80 nm, or said magnetic nanoparticle is in a shape of a cube.

8. The composition of claim 2, wherein said ethylene glycol is selected from the group consisting of monoethylene glycol, diethylene glycol, and polyethylene glycol, or a combination thereof.

9. The composition of claim 2, wherein said thiol is selected from the group consisting of thiotic acid, monothioctic acids, dithioctic acid and trithioctic acid, or a combination thereof.

10. The composition of claim 2, wherein said antibody is specific for a fetal trophoblast cell, a globin, or a circulating tumor cell.

11. A method of isolating a target cell or target biomolecule from a biological sample, said method comprising:
contacting said biological sample with a magnetic nanoparticle conjugated with an antibody via a linker to form a mixture, wherein said magnetic nanoparticle comprises a magnetic core and a non-magnetic outer surface layer, wherein said antibody is specific for said target cell or said target biomolecule, and wherein said linker comprises ethylene glycol and/or thiol;
incubating the mixture to covalently bind of the target cell or biomolecule to the antibody conjugated nanoparticle; and
separating the antibody conjugated nanoparticle that is bound to the target cell or biomolecule from the remainder of the biological sample using a magnetic field;
thereby isolating the target cell or biomolecule from the biological sample.

12. The method of claim 11, wherein said biological sample is obtained from a placenta or a vaginal swab of a pregnant woman, and wherein said target cell is a fetal trophoblast cell.

13. A method of detecting Down Syndrome in a fetus, the method comprising conducting a genetic test on one or more isolated fetal trophoblast cells obtained using the method of claim 12.

14. The method of claim 11, wherein said biological sample is a human feces sample, and wherein said target biomolecule is a human globin.

15. A method of detecting colorectal cancer in human, the method comprising conducting a colorimetric test on isolated human globin obtained using the method of claim 14.

16. The method of claim 11, wherein said biological sample is a human blood sample, and wherein said target cell is a circulating tumor cell, said circulating tumor cell being HER2 and EGFR positive metastatic cell.

17. The method of claim 16, wherein said antibody is Herceptin or Cetuximab.

18. A method of detecting lung cancer in a human, the method comprising conducting a genetic and fluorescent immunohistochemistry test on one or more isolated circulating tumor cells obtained using the method of claim 16.

19. A method of synthesizing an antibody magnetic nanoparticle conjugate, said method comprising:
mixing a magnetic nanoparticle with polyethylene glycol, wherein said magnetic nanoparticle comprises a magnetic core and a non-magnetic outer surface layer;
isolating PEGylated magnetic nanoparticle from the mixture;
covalently attaching one or more antibodies to the isolated PEGylated magnetic nanoparticle to form the antibody magnetic nanoparticle conjugate, wherein said antibodies are specific for one or more target cells or one or more target biomolecules that are to be isolated from a biological sample.

20. The method of claim 18, wherein said magnetic core comprises iron or iron oxide, and said non-magnetic outer layer comprises gold.

21. The method of claim 18, wherein said antibody is specific for a fetal trophoblast cell, a globin, or a circulating tumor cell.
